(19) United States
(12) Patent Application Publication
(10) Pub. No.: US 2014/0066966 A1
(43) Pub. Date: Mar. 6, 2014

(54) ENDOPYLORIC TOOL AND METHOD TO TREAT HYPERTROPIC PYLORIC STENOSIS

(71) Applicant: Children's National Medical Center, Washington, DC (US)

(72) Inventors: Peter C.W. KIM, Washington, DC (US); Timothy D. Kane, Washington, DC (US); Shannon McGue, Washington, DC (US); Axel Krieger, Washington, DC (US); Yonjae Kim, Washington, DC (US); Carolyn Cochenour, Washington, DC (US)

(73) Assignee: Children's National Medical Center, Washington, DC (US)

(21) Appl. No.: 14/015,402

(22) Filed: Aug. 30, 2013

Related U.S. Application Data
(60) Provisional application No. 61/695,184, filed on Aug. 30, 2012.

(51) Int. Cl. A61M 29/00

(52) U.S. Cl. CPC A61M 29/00
USPC 606/191

(57) ABSTRACT

A low profile gastrointestinal dilation catheter for use in infants includes a catheter tube, a dilation balloon, and at least one anchoring device. The dilation balloon is disposed over the catheter tube and is configured to transition between a compressed state and an expanded state. The at least one anchoring device is configured to prevent migration of the dilation balloon during a transition of the dilation balloon from the compressed state to the expanded state. A method to treat infantile hypertrophic pyloric stenosis (IHPS) is also provided.

### CROSS REFERENCE TO RELATED APPLICATION

This application is based upon and claims the benefit of priority from U.S. Provisional Patent Application No. 61/695,184, filed on Aug. 30, 2012, the entire contents of which are incorporated herein by reference.

### BACKGROUND

I. Field of the Disclosure

The present invention relates to medical care for relieving a stenosis. In particular, the invention relates to an inflatable balloon device for treating cases of hypertrophic pyloric stenosis in infants using dilation.

II. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Hypertrophic pyloric stenosis (HPS) is one of the most common reasons for infants to undergo surgery, occurring in approximately 10,000 births per year in the U.S. The pylorus is the muscle which separates the stomach from the duodenum, and in children with this condition, the muscular layers have become abnormally thickened. Correspondingly, the diameters of the pyloric canal and sphincter are reduced and thus do not permit normal passage of food. Infants present with progressive nonbilious vomiting, hunger, malnourishment, and dehydration. A physical exam should reveal a distended stomach and a palpable, olive-shaped mass in the epigastrium. The identification of this “olive” is considered diagnostic; if it is not detected, then the physician may order sonographic or endoscopic tests, or an upper GI series. Most commonly, patients develop HPS between 2-12 weeks of age. Currently, the stenosis is relieved by pyloromyotomy in which the surgeon makes a longitudinal incision of approximately 2 cm on the outside of the pylorus muscle. The muscular layers are completely cut and spread, while the sub-mucosal layer is left intact. This procedure was traditionally performed as an open surgery, but starting in 1991, laparoscopic pyloromyotomy began to be adopted as an alternative because of the improved cosmetic results.

Endoscopic balloon dilatation has been used to treat strictures in the digestive, respiratory, cardiovascular, excretory, and even reproductive systems of adults. It offers perfect cosmetic results, as well as the possibility for improved recovery rate, reduced hospital stay, and lower risk of complications. Furthermore, in some patients a transabdominal approach may be risky, and a method that uses only natural orifices would provide a viable alternative for them.

Pyloric balloon dilation is complicated by the tendency of the balloon to slide out of position during inflation. The high internal pressure of the hypertrophic pylorus will cause a conventional balloon (such as those used in angioplasty) to move proximally or distally into the relatively low pressure areas of the non-hypertrophic pylorus, the stomach, or the duodenum. It is known to use a dumbbell-shaped balloon in which the diameter of the central portion of the balloon is smaller than the proximal or distal portions, at least upon initial inflation. Such devices include U.S. Pat. No. 7,771,446 to Rutter (tracheal/bronchial dilation), U.S. Patent Publication No. 2007/0250104 to Condrea et al. (cervix), U.S. Pat. No. 7,951,111 to Drasler et al. (valvuloplasty), U.S. Pat. No. 5,947,991 to Cowan (cervix), U.S. Pat. No. 5,352,199 to Tower (valvuloplasty), and U.S. Pat. No. 6,488,653 to Lombardo (esophagus and intestine). However, none of these devices is specifically designed for pediatric use, and consequently these devices do not include the ability to compress to a diameter less than 2 mm. Narrow diameters would advantageously permit a catheter to be inserted through or alongside an endoscope, allowing for direct visualization of the site of dilation (e.g. the pylorus). Under a direct visualization, a physician could then immediately observe dilation, rupture, or any bleeding that might occur.

Other related art involves catheters which use a second distal balloon as an anchoring device. U.S. Patent Publication No. 2005/0055043 to Foltz et al. describes a device comprised of a dilation balloon and a second, distal anchoring balloon which are connected to two different catheter lumens in order to be inflated separately. This device is intended for application in cervical dilation. Disadvantages of balloon anchors include that the balloon anchor may have to be inflated to a very large diameter, that it may be difficult to compress back to a slim profile after a large inflation, and that its effectiveness will decrease as the circumference at the site of dilation increases, as it inevitably will during dilation. Taking dilation of hypertrophic pyloric stenosis as an example, the desired diameter of the pyloric channel after dilation is 4 to 8 mm, which is much greater than the diameter of the pyloric channel in hypertrophic pyloric stenosis, which ranges from 1 to 4 mm. An anchoring balloon would need to inflate to a diameter greater than the diameter at the end of dilation, in order to prevent migration throughout the dilation procedure. Both the challenges of delivering sufficient pressure and preventing slippage should be met, however.

### SUMMARY

Considering the potential advantages of endoscopic pyloric balloon dilation and the aforementioned difficulties associated with conventional techniques, there is a need to develop an innovative balloon tool and procedure for using this tool to dilate a pyloric stenosis in young infants. This tool should be highly compressible from its inflated diameter, resist axial slippage, and provide enough force to achieve complete pyloric dilation. These characteristics may be useful in balloon dilation of strictures located in other areas of the body, particularly in areas accessible only to small endoscopes or areas where balloons are prone to slippage upon inflation.

It is therefore a primary object of the present invention to provide a catheter-based balloon dilator for medical application that provides careful, controlled, and directed dilating pressure to a site of stenosis or stricture.

It is an object of the present invention to provide a low profile catheter-based dilator, including but not limited to a balloon-based dilator, wherein a design allows the passage through a working channel of an endoscope 2 mm or less.

It is an object of the present invention to provide a catheter-based balloon dilator including a low profile design that allows the passage of a balloon catheter through the narrow pyloric channel <2 mm.

It is an object of the present invention to provide a catheter-based balloon dilator including an anchoring device to prevent migration of the tool.

Another object of the present invention is to provide a catheter-based balloon dilator in which the balloon and anchoring device can be compacted to a low diameter for insertion through narrow bodily passageways.

Yet another object of the present invention is to provide a dilation balloon with a working length between 15 to 25 mm, and preferably 25 mm for the application of infantile hypertrophic pyloric stenosis (IHPS).

Another object of the present invention is to provide a dilation balloon with an expanded diameter of 4 to 8 mm, and preferably 6 mm for the application of IHPS.

Yet another object of the present invention is to provide a catheter stiff enough to protrude through a small lumen while meeting a minimum force requirement of 200 gf (grams force) exerted without kinking or buckling.

It is also an object of the present invention to deploy a catheter-based balloon dilator while under visualization.

Another object of the present invention is to provide a cyclic dilation sequence to dilate a hypertrophic lumen, such as but not limited to three cycles of two minute inflations.

Yet another object of the present invention is to provide a method for using this catheter-based balloon dilator to perform dilation of a stenosis or stricture in pediatric populations.

These objectives, among others, are attained by the present invention described in this disclosure.

In an aspect of the disclosure, a low profile gastrointestinal dilation catheter for use in infants includes a catheter tube, a dilation balloon, and at least one anchoring device. The dilation balloon is disposed over the catheter tube and is configured to transition between a compressed state and an expanded state. The at least one anchoring device is configured to prevent migration of the dilation balloon during a transition of the dilation balloon from the compressed state to the expanded state.

In an aspect of the disclosure, the low profile gastrointestinal dilation catheter includes an outermost diameter <2 mm in the compressed state.

In an aspect of the disclosure, the at least one anchoring device includes a distal stabilizing device. The distal stabilizing device is a balloon or an umbrella-like structure.

In an aspect of the disclosure, the at least one anchoring device includes a proximal stabilizing device. The proximal stabilizing device is a balloon or an umbrella-like structure.

In an aspect of the disclosure, the catheter tube is disposed over a guide wire, or alternatively, the catheter tube may not be disposed over a guide wire.

In an aspect of the disclosure, the catheter tube is disposed through an endoscope.

In an aspect of the disclosure, the transition of the dilation balloon from the compressed state to the expanded state is visualized through an endoscope or with another internal visualization device.

In an aspect of the disclosure, the transition of the dilation balloon from the compressed state to the expanded state is visualized by an external viewing modality.

In an aspect of the disclosure, the catheter is placed through a natural orifice of a patient's body.

In an aspect of the disclosure, the catheter tube includes a plurality of lumens.

In an aspect of the disclosure, the catheter tube includes a stiffness that varies along a length of the catheter tube.

In an aspect of the disclosure, the at least one anchoring device is a stabilizing balloon, and the dilation balloon is made of a first material and the stabilizing balloon is made of a second material that includes a durometer different than a durometer of the first material.

In an aspect of the disclosure, the dilation balloon is a high pressure balloon that is inflatable to pressures ranging from 2 to 20 atm, and preferably 14 to 20 atm.

In an aspect of the disclosure, the at least one anchoring device is a stabilizing balloon that is made of a compliant material.

Another aspect of the disclosure includes a method for treating infantile hypertrophic pyloric stenosis (IHPS) including using the low profile gastrointestinal dilation catheter.

A further aspect of the disclosure includes a method to treat infantile hypertrophic pyloric stenosis (IHPS). The method includes placing a catheter into a stomach of a patient. The method includes passing the catheter through a pyloric channel. The method includes deploying a distal stabilizing device of the catheter in a duodenum. The method includes inflating a dilation balloon of the catheter in the pyloric channel. The method includes deflating the dilation balloon and compacting a dimension of the distal stabilizing device. The method also includes retracting the catheter through a natural orifice of the patient.

In an aspect of the disclosure, the method includes pulling back on the distal stabilizing device with the catheter before inflating the dilation balloon.

In an aspect of the disclosure, the method includes placing a guide wire through the catheter and advancing the guide wire through the pyloric channel prior to insertion of the catheter into the pyloric channel.

In an aspect of the disclosure, the method includes deploying a proximal stabilizing device of the catheter in an antrum of the stomach prior to inflating the dilation balloon. The proximal stabilizing device is a balloon that is inflated in the antrum of the stomach, or an umbrella-like structure that is deployed in the antrum of the stomach.

In an aspect of the disclosure, the method includes the distal stabilizing device being a balloon that is inflated in the duodenum, or an umbrella-like structure that is deployed in the duodenum.

In an aspect of the disclosure, the method includes inserting the catheter through a mouth of the patient's body.

In an aspect of the disclosure, the method includes advancing the catheter through a working channel of an endoscope, or advancing the catheter alongside an endoscope.

In an aspect of the disclosure, the inflation of the dilation balloon includes an inflation cycle of a plurality of inflations of the dilation balloon with a period of rest or deflation of the dilation balloon between a first inflation and a second inflation.

In an aspect of the disclosure, the method includes visualizing placement of the catheter through an endoscope.

In an aspect of the disclosure, the method includes visualizing placement of the catheter using ultrasound.

In an aspect of the disclosure, the method includes visualizing placement of the catheter under fluoroscopy or with a visualization method using radiation.

In an aspect of the disclosure, the method includes visualizing placement of the catheter under magnetic resonance (MR) or with a visualization method not using radiation.

The foregoing paragraphs have been provided by way of general introduction, and are not intended to limit the scope of the following claims. The described embodiments, together with precise advantages, will be best understood by reference to the following detailed description taken in conjunction with the accompanying drawings.

### BRIEF DESCRIPTION OF THE DRAWINGS

A more complete appreciation of the disclosure and the attendant advantages thereof will be better understood by reference to the accompanying drawings and the subsequent detailed description, where:

FIG. 1 depicts a schematic view of a child with infantile hypertrophic pyloric stenosis (IHPS).

FIG. 2 depicts a low profile catheter including a balloon based dilator and a distal stabilizing device, in a close-up cross-sectional view of proximal and distal ends (top), and in a full side view of the entire catheter (bottom).

FIG. 3 depicts a cross-sectional view of the distal end of the low profile catheter including the balloon based dilator and the distal stabilizing device.

FIG. 4 depicts a cross-sectional view revealing a multi-lumen aspect of the low profile catheter.

FIG. 5A depicts a variation of the low profile catheter including a balloon based dilator and both distal and proximal stabilizing devices shown as balloons.

FIG. 5B depicts a variation of the low profile catheter including a balloon based dilator and a proximal stabilizing device shown as a balloon.

FIG. 5C depicts a variation of the low profile catheter including a balloon based dilator without any stabilizing devices.

FIG. 6A depicts a variation of the low profile catheter including a balloon based dilator and both distal and proximal stabilizing devices shown as balloons, in which the low profile catheter is placed through an endoscope and into the pylorus.

FIG. 6B depicts a variation of the low profile catheter including a balloon based dilator and a proximal stabilizing device shown as a balloon, in which the low profile catheter is placed through an endoscope and into the pylorus.

FIG. 6C depicts a variation of the low profile catheter including a balloon based dilator with no stabilizing device, in which the low profile catheter is placed through an endoscope and into the pylorus.

FIG. 7A depicts a variation of the low profile catheter including a balloon based dilator and both distal and proximal stabilizing devices shown as umbrella-like structures.

FIG. 7B depicts a variation of the low profile catheter including a balloon based dilator and a distal stabilizing device shown as an umbrella-like structure.

FIG. 7C depicts a variation of the low profile catheter including a balloon based dilator and a proximal stabilizing device shown as an umbrella-like structure.

FIG. 8A depicts a variation of the low profile catheter including a balloon based dilator and both distal and proximal stabilizing devices shown as umbrella-like structures, in which the low profile catheter is placed through an endoscope and into the pylorus.

FIG. 8B depicts a variation of the low profile catheter including a balloon based dilator and a distal stabilizing device shown as an umbrella-like structure, in which the low profile catheter is placed through an endoscope and into the pylorus.

FIG. 8C depicts a variation of the low profile catheter including a balloon based dilator and a proximal stabilizing device shown as an umbrella-like structure, in which the low profile catheter is placed through an endoscope and into the pylorus.

FIG. 9A depicts a completion of method steps of inserting the endoscope through the mouth and into the stomach of a child patient to visualize the pyloric channel, passing a catheter through the pyloric channel, and then inflating a distal stabilizing device in the duodenum.

FIG. 9B depicts a completion of a method step of pulling back on an inflated distal stabilizing balloon to align a dilation balloon with a hypertrophied pyloric muscle and to anchor the catheter.

FIG. 9C depicts a completion of a method step of inflating the dilation balloon to expand the hypertrophied pyloric muscle.

FIG. 9D depicts a completion of method steps of deflating both the dilation balloon and the distal stabilizing balloon, retracting the catheter through the endoscope, and then removing the catheter and the endoscope from the child patient. After the dilation procedure is completed, the pylorus now allows food to pass normally.

### DETAILED DESCRIPTION OF THE ILLUSTRATIVE EMBODIMENTS

Referring to the drawings, like reference numerals designate identical or corresponding parts throughout the several views.

### I. Introduction

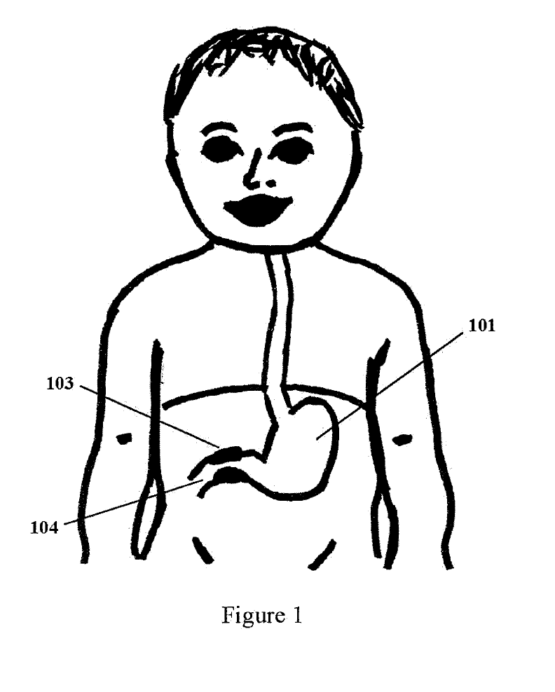

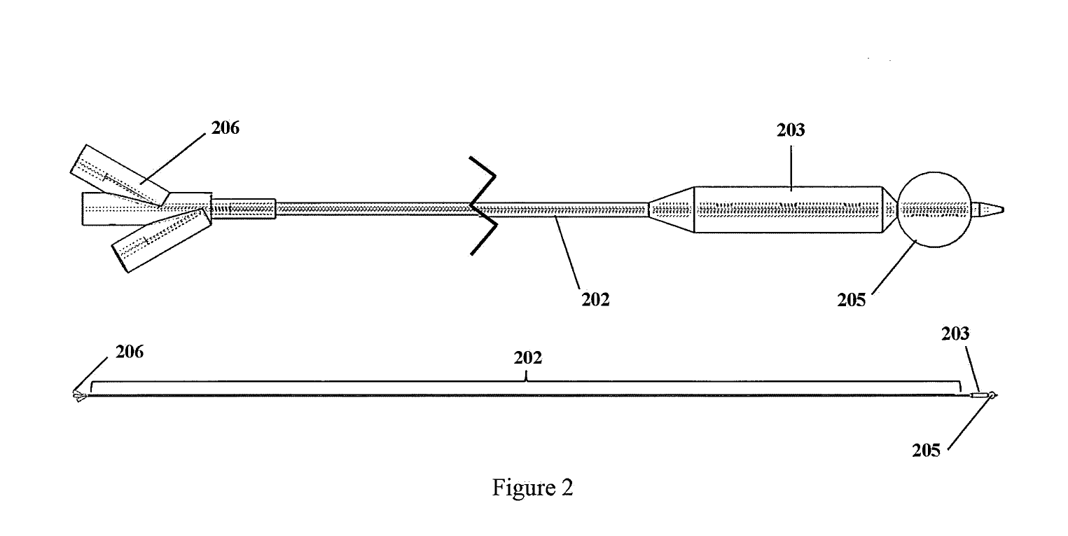

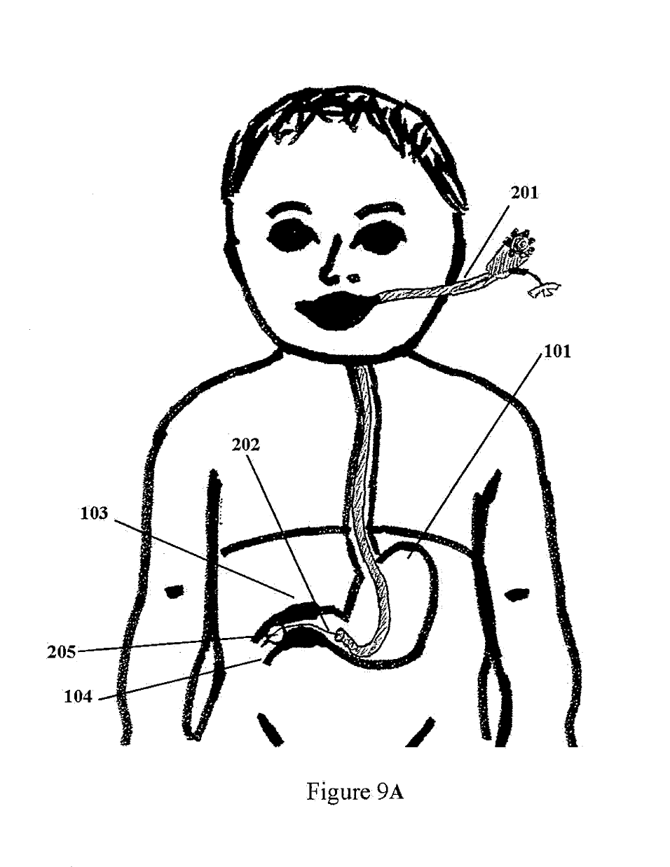

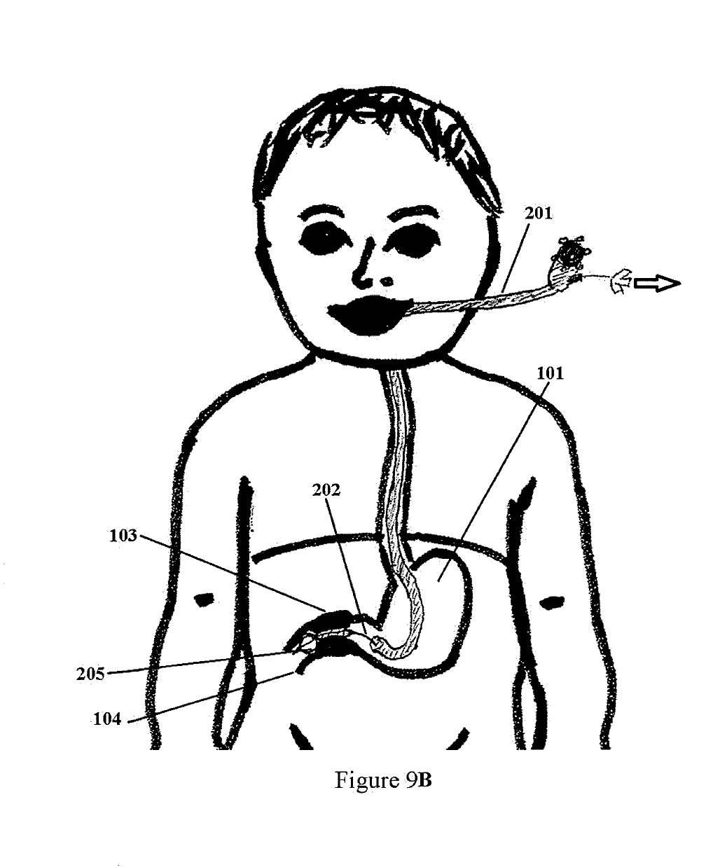

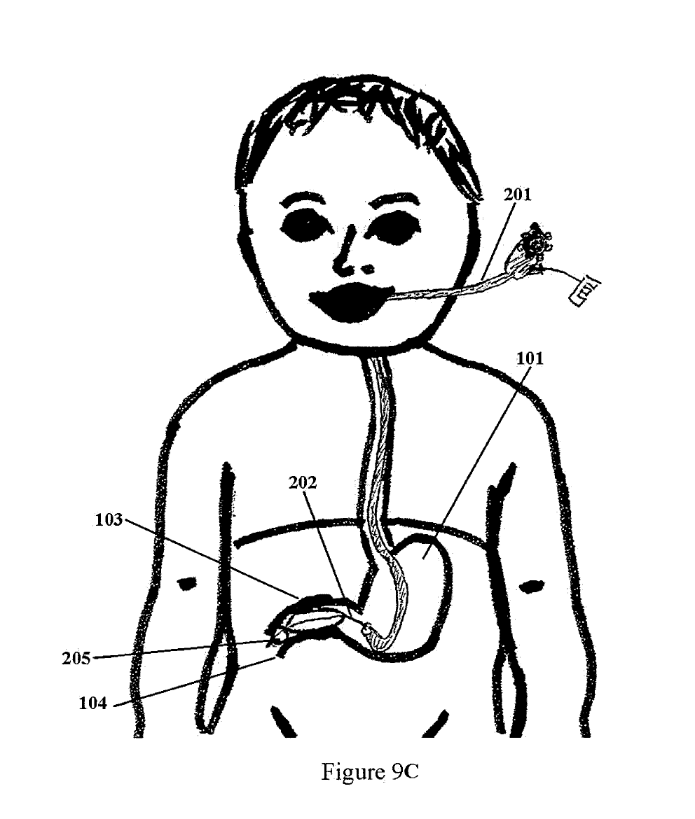

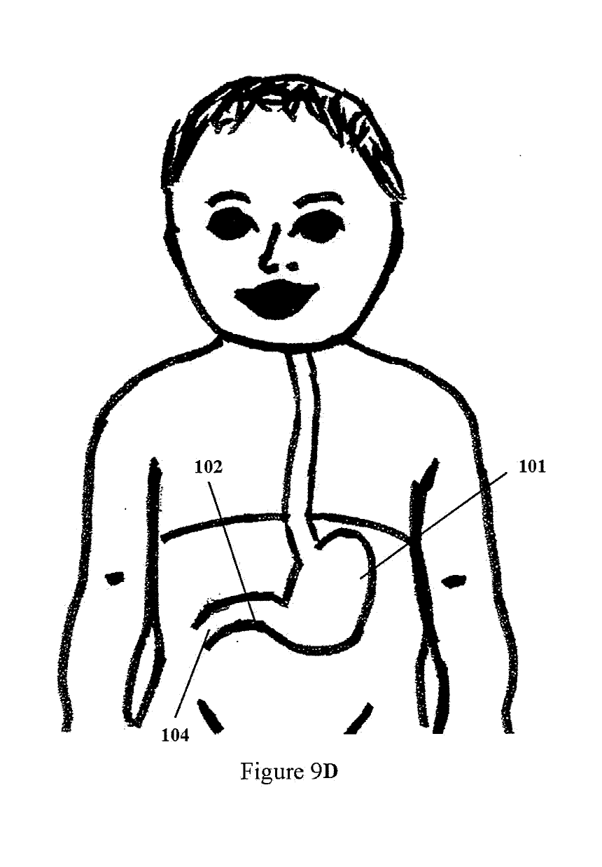

The present invention generally involves a catheter-based apparatus and methods for treatment of infantile hypertrophic pyloric stenosis (IHPS). Referring the FIG. 1, IHPS is the enlargement of the pyloric muscle 103 between the stomach 101 and the small intestine 104, and is apparent in certain infants younger than 6 months. FIG. 1 demonstrates the relevant anatomy for reference. FIGS. 2-8 show illustrative, but not all variations of the present invention. Meanwhile, FIGS. 9A-D illustrate an example of a method of novel treatment for IHPS.

### II. Low Profile Gastrointestinal Dilation Catheter Apparatus

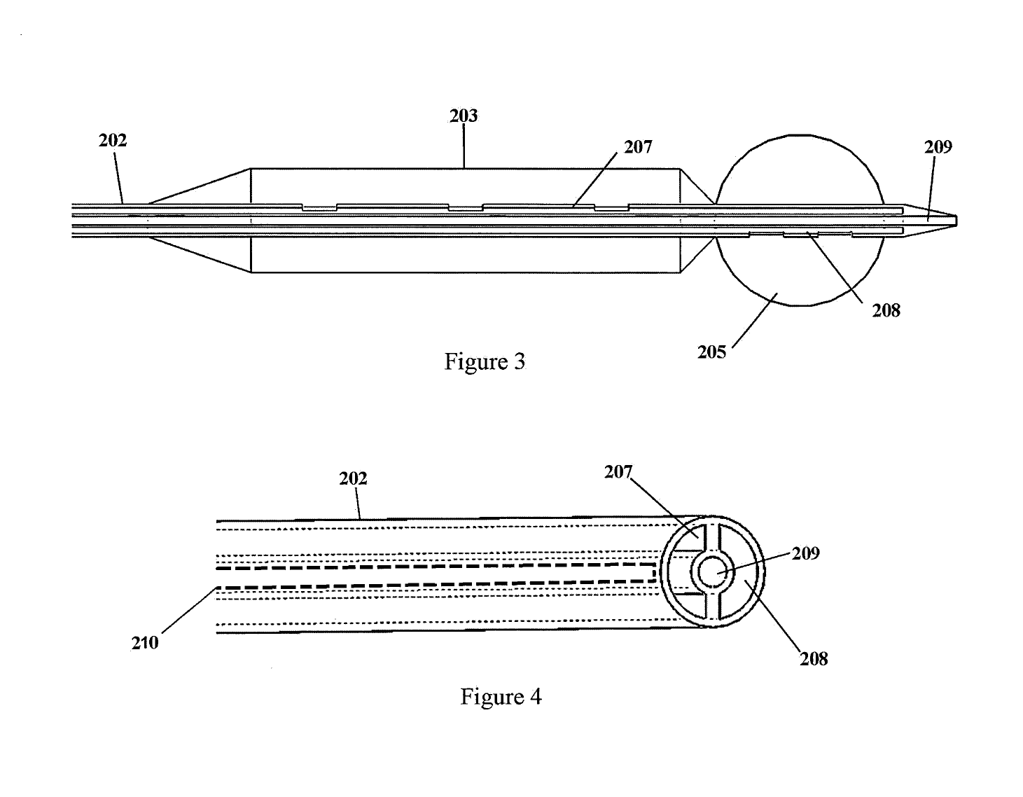

The low profile gastrointestinal dilation catheter apparatus includes four general components as shown in FIG. 2: (1) a dilation catheter 202, (2) a dilation balloon 203, (3) a stabilizing device 205, and (4) a manifold 206. Cross sections of the apparatus are illustrated in FIGS. 3 and 4. The present invention involves a multi-lumen catheter tube including at least two lumens 207-209, at least one of which may be designated for insertion of a guide wire 210. One or more connections between the dilation catheter 202 and an inner cavity of each of the dilation balloon 203 and any stabilizing device(s) 204-205 permit gas or liquid transported from a reservoir to inflate and deflate the dilation balloon and stabilizing device(s). In one example, expansion and compression of the dilation balloon 203 and any stabilizing device(s) 204-205 are triggered by an outside control apparatus operated by a surgeon, and the control apparatus is connected to the dilation catheter apparatus via one or more lumens of the catheter tube. The dilation balloon 203 and stabilizing device(s) 204-205 in one embodiment are each connected to separate lumens of the catheter tube. This invention may be used with or without a guide wire. If a guide wire is used, the dilation catheter apparatus is tracked over the guide wire in a concentric fashion. The dilation catheter 202 may be made from a variety of plastics such as but not limited to: Pebax®, nylon, or polyimide. Additionally, portions of the catheter may contain a braided wire structure to increase the stiffness of the dilation catheter 202, particularly at the distal end where the catheter advances through the hypertrophic pylorus 103. The dilation catheter 202 is stiff enough to protrude through a small lumen while meeting a minimum force requirement of 200 gf (grams force) exerted without kinking or buckling.

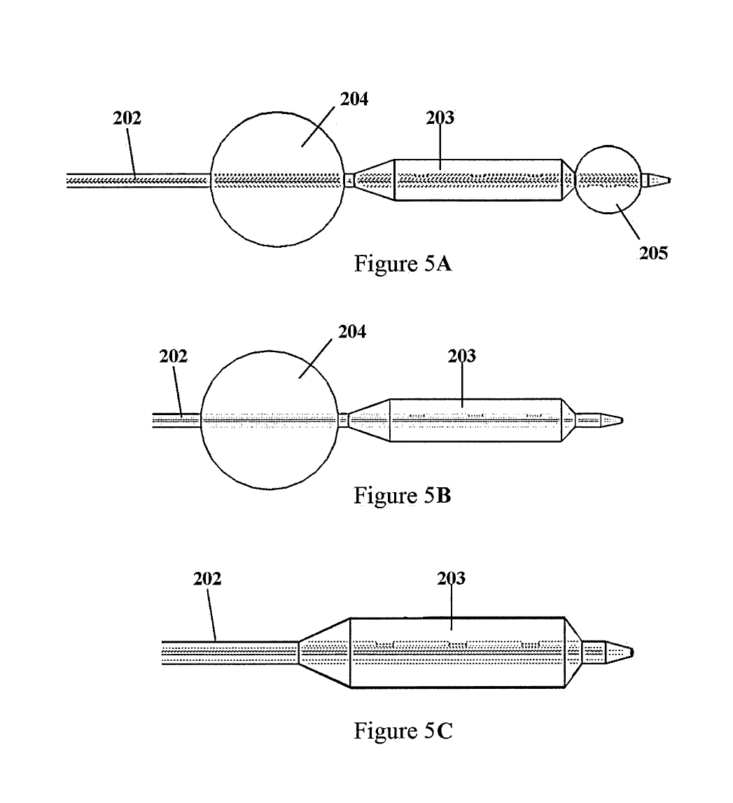

The dilation balloon 203 has a main purpose of providing force circumferentially to expand the lumen of the hypertrophic pylorus 103. The diameter of the hypertrophic pylorus generally ranges from 1 to 4 mm. The desired diameter of the dilation balloon 203 after dilation is approximately 2.5 to 3 times an initial lumen diameter, and preferably a dilated diameter of 4 to 8 mm. Therefore, based on averages, it is advantageous to dilate the dilation balloon 203 to 6 mm for the application of infantile hypertrophic pyloric stenosis (IHPS). The dilation balloon 203 is cylindrical in shape with minimal taper on the distal portion. The dilation balloon may also embody other shapes such as but not limited to a wedge or conical shape. A working length of the dilation balloon 203 is between 15 to 25 mm, and preferably 25 mm for the application of IHPS. One embodiment of the apparatus includes just the dilation balloon 203 on the catheter without any stabilizing device (FIG. 5C). The stabilizing device(s) may be supplied by a separate balloon catheter housing the stabilizing device(s) on which the dilation balloon catheter tracks.

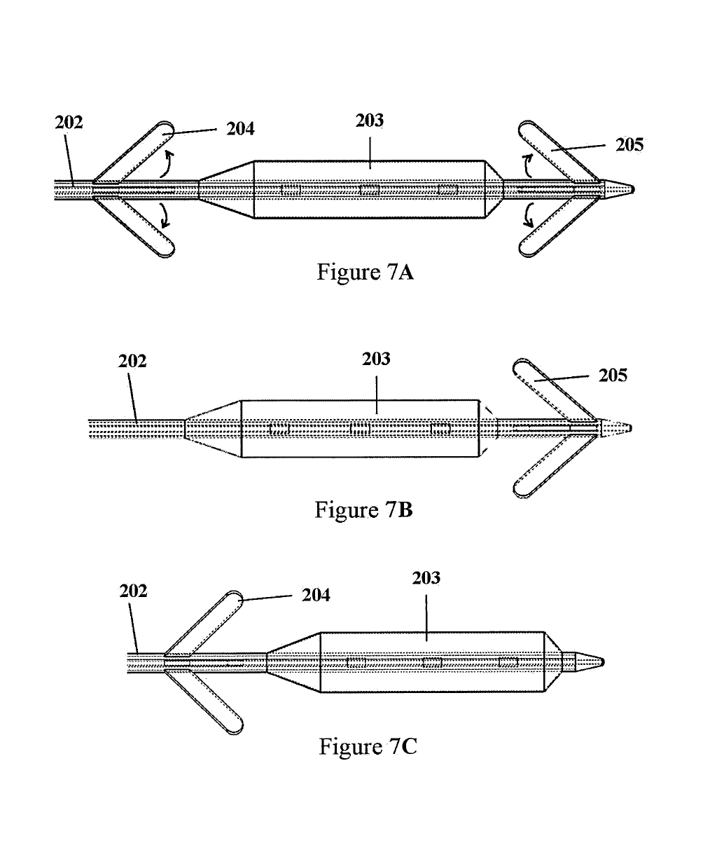

Additionally, the stabilizing device(s) may be present as, but not limited to balloons (FIG. 5A-C) or umbrella-like structure(s) (FIGS. 7A-C). These stabilizing or anchoring devices are deployed before the dilation balloon is inflated, to avoid slippage of the dilation balloon due to a pressure caused by the inflating dilation balloon. The dilation balloon, in one example, is a high pressure balloon that is inflatable to pressures ranging from 2 to 20 atm, and preferably 14 to 20 atm.

The balloon type stabilizing device(s) is shown in multiple embodiments in FIGS. 5A-C. These stabilizing balloons 204, 205 may be made from but not limited to compliant materials including plastics such as urethane or silicone. These balloons 204, 205 serve the purpose to prevent migration of the dilation balloon 203 as well as to protect the healthy areas of tissue, such as the duodenum. A proximal stabilizing balloon 204 may be larger than a distal stabilizing balloon 205 and vice versa. The shape of these balloons may also be varied. FIGS. 5A-B show spherical balloons, however, other embodiments include, but are not limited to disk-like, conical or square shapes. FIG. 5A shows both proximal and distal stabilizing balloons.

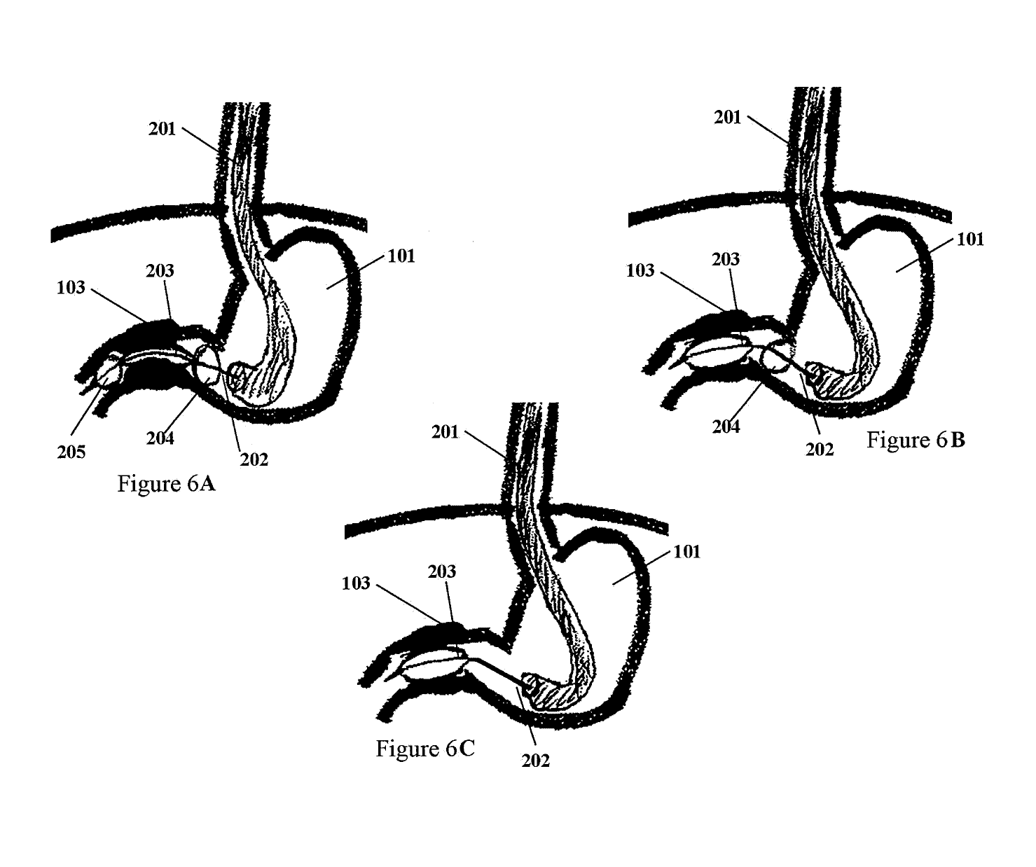

FIG. 6A shows how this embodiment of the apparatus would be deployed into the hypertrophic pylorus 103. In this configuration, the balloons are inflatable independent of each other or together. As demonstrated in FIG. 6A, the two stabilizing balloons 204, 205 are placed on either side of the hypertrophied pyloric muscle 103. The distal stabilizing balloon sits against the duodenum wall and the proximal stabilizing balloon sits against the antrum wall at the base of the stomach. This catheter is intended to be used through a working channel of an endoscope 201.

FIG. 5B shows only a proximal stabilizing balloon 204.

FIG. 6B shows how this embodiment of the apparatus would be deployed into the hypertrophic pylorus 103. In this configuration the balloon is inflatable independent of the dilation balloon or together. As demonstrated in FIG. 6B, the proximal stabilizing balloon 204 is placed in the antrum of the stomach. The proximal stabilizing balloon 204 sits against the antrum wall at the base of the stomach. This catheter is intended to be used through a working channel of an endoscope 201.

The umbrella type stabilizing device(s) is shown in multiple embodiments in FIGS. 7A-C. These stabilizing umbrellas may be made from but not limited to flexible plastics, rigid plastics, shape memory alloys, or metals. The umbrella devices serve the purpose to prevent migration of the dilation balloon as well as to protect the healthy areas of tissue, such as the duodenum. The proximal stabilizing umbrella 204 may be larger than the distal stabilizing umbrella 205 and vice versa. Each umbrella contains at least two arms which may be in the form of straight arms, jointed arms, curved arms, or any other geometry that would serve the function of stabilizing the dilation balloon 203. Ends of the arms are preferably blunt or rounded to decrease the risk of the arms from puncturing or causing other injury to a body passageway, and friction between the ends of the arms and the body passageway prevents migration of the apparatus. A material may also cover the arms to further prevent injury. FIGS. 7A-B show straight armed umbrella devices 204, 205. FIG. 7A shows both proximal and distal stabilizing umbrellas 204, 205, with arrows depicting deployment directions of the arms of each umbrella. One or more springs joined to the arms and/or one or more control wires extending through the catheter and outside the patient's body, are used to expand and contract the umbrella devices 204, 205 for example, although any other activating/deactivating mechanism or means may be used.

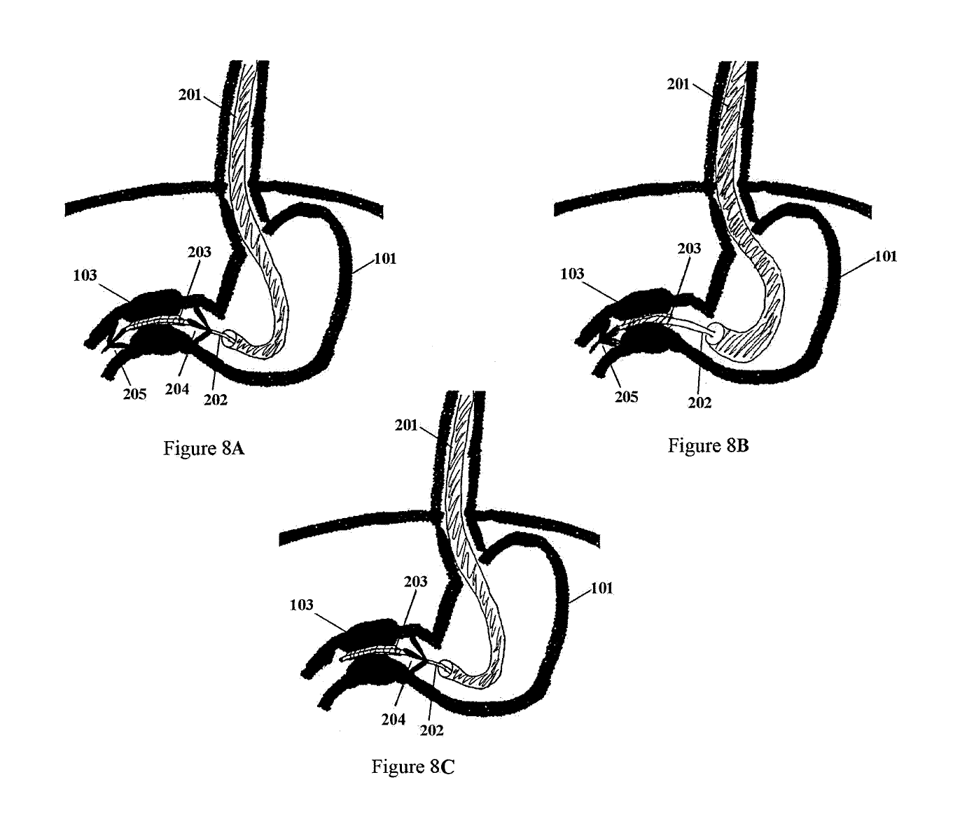

FIG. 8A shows how this embodiment of the apparatus would be deployed into the hypertrophic pylorus 103. In this configuration the umbrella devices are deployable independent of each other or together. As demonstrated in FIG. 8A, the two stabilizing umbrella devices 204, 205 are placed on either side of the hypertrophied pyloric muscle 103. The distal stabilizing umbrella 205 sits against the duodenum wall and the proximal stabilizing umbrella 204 sits against the antrum wall at the base of the stomach. This catheter is intended to be used through a working channel of an endoscope 201.

FIG. 7B shows only a distal stabilizing umbrella 205. FIG. 8B shows how this embodiment of the apparatus would be deployed into the hypertrophic pylorus 103. In this configuration the umbrella is deployable independent of the dilation balloon 203 or together. As demonstrated in FIG. 8B, the distal stabilizing umbrella is placed in the duodenum. The distal stabilizing umbrella 204 sits against the duodenum wall behind the hypertrophic pylorus 103. This catheter is intended to be used through a working channel of an endoscope 201.

FIG. 7C shows only a proximal stabilizing umbrella 204. FIG. 8C shows how this embodiment of the apparatus would be deployed into the hypertrophic pylorus 103. In this configuration the umbrella is deployable independent of the dilation balloon 203 or together. As demonstrated in FIG. 8C, the proximal stabilizing umbrella 204 is placed in the antrum of the stomach. The proximal stabilizing umbrella 204 sits against the antrum wall at the base of the stomach. This catheter is intended to be used through a working channel of an endoscope 201.

### III. Method for Treatment of Infantile Hypertrophic Pyloric Stenosis (IHPS)

FIGS. 9A-D show a method of treatment of hypertrophic pyloric stenosis using the invention described above. One aspect in which this method is unique is that it allows for the treatment of IHPS through a natural orifice such as the mouth. The method begins by a physician inserting a gastric endoscope 201 through the mouth and into the stomach 101 of the patient. Here the physician identifies visually the hypertrophic pyloric channel 103. The balloon dilation catheter apparatus is then fed through a working channel of the endoscope 201 and threaded through the hypertrophic pyloric channel 103. The physician can either use a guide wire first to pass through the hypertrophic pylorus 103 and then track the balloon dilation catheter over the guide wire or the physician can do this without a guide wire. FIG. 9A illustrates this step. Once the balloon dilation catheter is beyond the hypertrophic pylorus 103 the physician engages the distal stabilizing device 204 either by inflating a balloon or deploying an umbrella. The physician may then pull the distal stabilizing device 204 back flush against the distal portion of the hypertrophic pylorus 103 to precisely locate the dilation balloon 203 as shown in FIG. 9B. If there is a proximal stabilizing device 205, it is inflated or deployed proximal to the hypertrophic pylorus 103 in the antrum of the stomach at this point. Once the balloon dilation catheter 202 is fixed, the physician can inflate the dilation balloon 203 as shown in FIG. 9C. The dilation balloon 203 at this point should be positioned properly due to the earlier positioning of any stabilizing device(s). The dilation balloon 203 is inflated for a minimum of thirty seconds and may be repeated any number of times to the physician's satisfaction. For example, it is possible to take a cyclic approach with a plurality of dilations by inflating the dilation balloon 203 for two minutes followed by a period of rest or deflation, then followed by two additional inflation cycles of two minutes each that are each followed by a period of rest or deflation. The number of inflations and periods of rest or deflation is not limited to three cycles, however, and any number of cycles may be employed. Once the dilation of the hypertrophic pylorus 103 is complete, the dilation balloon 203 is deflated and each stabilizing device is deflated or retracted. The balloon dilation catheter is then withdrawn into the working channel of the endoscope 201 and removed from the patient. Finally, the endoscope is removed from the patient. As shown in FIG. 9D, the patient is clear of all devices and the pylorus 102 is now dilated.

While this method describes specifically treatment of IHPS using the balloon dilation catheter, the present invention may be used to dilate any region or passageway reachable via a natural orifice of a patient's body. More generally one or more stabilizing devices would be placed on either side of a lesion, or alternatively, only on one side. With the dilation balloon stabilized at or near the area of the stenosis, the dilation balloon is inflated, expanding the stenosis. After the dilation, the stabilizing devices and dilation balloon are deflated and the entire system retracted through the working channel of an endoscope. Finally the endoscope is removed from the patient leaving the stenosis no longer present.

The foregoing disclosure describes merely illustrative embodiments of the present invention. As will be understood by those skilled in the art, the present invention may be embodied in other specific forms without departing from the spirit or essential characteristics thereof. Accordingly, the disclosure is intended to be illustrative of the present invention, but not limiting of the scope of the invention, as well as the following claims. The disclosure and any discernible variants of the teachings herein define, at least in part, the scope of the claim terminology, such that no inventive subject matter is dedicated to the public.

1. A low profile gastrointestinal dilation catheter for use in infants, comprising:
a catheter tube;
a dilation balloon that is disposed over the catheter tube and is configured to transition between a compressed state and an expanded state; and
at least one anchoring device that is configured to prevent migration of the dilation balloon during a transition of the dilation balloon from the compressed state to the expanded state.

2. The low profile gastrointestinal dilation catheter according to claim 1, wherein the low profile gastrointestinal dilation catheter includes an outermost diameter <2 mm in the compressed state.

3. The low profile gastrointestinal dilation catheter according to claim 1, wherein the at least one anchoring device includes a distal stabilizing device that is a balloon or an umbrella-like structure.

4. The low profile gastrointestinal dilation catheter according to claim 1, wherein the at least one anchoring device includes a proximal stabilizing device that is a balloon or an umbrella-like structure.

5. The low profile gastrointestinal dilation catheter according to claim 1, wherein the catheter tube includes a plurality of lumens.

6. The low profile gastrointestinal dilation catheter according to claim 1, wherein the catheter tube includes a stiffness that varies along a length of the catheter tube.

7. The low profile gastrointestinal dilation catheter according to claim 1, wherein the at least one anchoring device is a stabilizing balloon, and the dilation balloon is made of a first material and the stabilizing balloon is made of a second material that includes a durometer different than a durometer of the first material.

8. The low profile gastrointestinal dilation catheter according to claim 1, wherein the dilation balloon is a high pressure balloon that is inflatable to pressures ranging from 2 to 20 atm.

9. A method for treating infantile hypertrophic pyloric stenosis (IHPS), comprising:
using the low profile gastrointestinal dilation catheter according to claim 1.

10. A method to treat infantile hypertrophic pyloric stenosis (IHPS), comprising:
placing a catheter into a stomach of a patient;
passing the catheter through a pyloric channel;
deploying a distal stabilizing device of the catheter in a duodenum;
inflating a dilation balloon of the catheter in the pyloric channel;
deflating the dilation balloon and compacting a dimension of the distal stabilizing device; and
retracting the catheter.

11. The method to treat infantile hypertrophic pyloric stenosis (IHPS) according to claim 10, further comprising:
pulling back on the distal stabilizing device with the catheter before inflating the dilation balloon.

12. The method to treat infantile hypertrophic pyloric stenosis (IHPS) according to claim 10, further comprising:
placing a guide wire through the catheter and advancing the guide wire through the pyloric channel prior to insertion of the catheter into the pyloric channel.

13. The method to treat infantile hypertrophic pyloric stenosis (IHPS) according to claim 10, wherein the inflating the dilation balloon includes an inflation cycle of a plurality of inflations of the dilation balloon with a period of rest or deflation between inflations of the dilation balloon.

14. The method to treat infantile hypertrophic pyloric stenosis (IHPS) according to claim 10, further comprising:
deploying a proximal stabilizing device of the catheter in an antrum of the stomach prior to inflating the dilation balloon.

15. The method to treat infantile hypertrophic pyloric stenosis (IHPS) according to claim 14, wherein the proximal stabilizing device is a balloon that is inflated in the antrum of the stomach, or an umbrella-like structure that is deployed in the antrum of the stomach.

16. The method to treat infantile hypertrophic pyloric stenosis (IHPS) according to claim 10, wherein the distal stabilizing device is a balloon that is inflated in the duodenum.

17. The method to treat infantile hypertrophic pyloric stenosis (IHPS) according to claim 10, wherein the distal stabilizing device is an umbrella-like structure that is deployed in the duodenum.

18. The method to treat infantile hypertrophic pyloric stenosis (IHPS) according to claim 10, further comprising:
inserting and extracting the catheter through a natural orifice of the patient's body.

19. The method to treat infantile hypertrophic pyloric stenosis (IHPS) according to claim 10, further comprising:
advancing the catheter through a working channel of an endoscope, or alongside the endoscope.

20. The method to treat infantile hypertrophic pyloric stenosis (IHPS) according to claim 10, further comprising:
visualizing placement of the catheter using at least one of ultrasound, fluoroscopy, a visualization method using radiation, magnetic resonance (MR), and an endoscope.
